SEQUENCE LISTING (2 sequences)

SEQ ID NO: 1 - DNA, 21 bp, Artificial Sequence
  Synthetic
  modified_base (6)..(6): RNA nucleobase
  modified_base (14)..(14): RNA nucleobase
  modified_base (20)..(20): RNA nucleobase
  modified_base (21)..(21): 35OctdU (co5-Octadiynyl dU)
gccccatcaugtgcctttccu

SEQ ID NO: 2 - DNA, 21 bp, Artificial Sequence
  Synthetic
  modified_base (6)..(6): RNA nucleobase
  modified_base (18)..(18): i5OctdU (5-Octadiynyl dU)
gccccatcaugtgcctuucct

(19) United States
(12) Patent Application Publication
(10) Pub. No.: US 2024/0247302 A1
(43) Pub. Date: Jul. 25, 2024

(54) MASSIVE GENERATION OF CHEMICALLY LIGATEABLE PROBES FOR MULTIPLEXED FISH

(71) Applicant: CALIFORNIA INSTITUTE OF TECHNOLOGY, Pasadena, CA (US)

(72) Inventors: Long CAI, Pasadena, CA (US); Chee Huat (Linus) ENG, Pasadena, CA (US)

(21) Appl. No.: 18/564,478

(22) PCT Filed: May 27, 2022
(86) PCT No.: PCT/US22/31458

§ 371 Date: Nov. 27, 2023

Related U.S. Application Data
(60) Provisional application No. 63/194,511, filed on May 28, 2021.

(51) Int. Cl. C12Q 1/6806

(52) U.S. Cl. CPC C12Q 1/6806

(57) ABSTRACT

Disclosed herein are methods for generating a massive number of chemically ligateable probes for multiplexed Fluorescence In Situ Hybridization (FISH) using a hybrid-primer. Also, the disclosure sets forth methods, in addition to using the same, and other solutions to problems in the relevant field.

### CROSS-REFERENCE TO RELATED APPLICATIONS

This application claims the benefit of U.S. Provisional Application Ser. No. 63/194,511 filed May 28, 2021, the entirety of which is incorporated herein by reference.

### BACKGROUND

Combination of in vitro transcription and reverse transcription has been used to generate single stranded probes. These probes are used for direct detection of molecules in cells. However, in order to generate chemically ligateable probes, two ligateable moieties are often placed on the oligo such that when they hybridize on the target, the two functional groups are placed within molecular distances of each other to allow the ligation to occur.

To do so, the current methods involve in-tube ligation with DNA ligase to ligate multiple fragments of oligonucleotides with one or multiple fragments of the oligonucleotides containing the functional groups suitable for chemical ligation synthesized by solid-phase synthesis. However, this method is cost prohibitive to scale up beyond a few probes. In genomic applications, often hundreds to millions of such probes are needed.

### SUMMARY

The present disclosure provides a method for generating a massive number of chemically ligateable probes for multiplexed Fluorescence In Situ Hybridization (FISH). This disclosure sets forth methods, in addition to using the same, and other solutions to problems in the relevant field.

In some embodiments, there is provided a method to generate probes, the method comprising: contacting one or more RNA templates with a reverse transcriptase and one or more hybrid-primers under conditions suitable for reverse transcription, wherein each hybrid-primer hybridizes to at least one of the one or more RNA templates. In some embodiments, each hybrid-primer comprises one or more deoxyribonucleotides. In certain embodiments, each hybrid-primer comprises one or more ribonucleotides. In certain embodiments, each hybrid-primer comprises one or more reactive groups, at the 3′ end of the primer. In some embodiments, the method comprises degrading the RNA template and the hybrid-primer. In some embodiments, the method comprises isolating one or more single stranded DNA probes with at least of the one or more reactive groups at its 5′ end.

In some embodiments, the methods are used to generate probes for use in an efficient and scalable signal amplification method that can be applied to multiplexed imaging. In certain embodiments, the methods are used to generate probes for use in RNA and DNA sequential Fluorescence In Situ Hybridization (seqFISH). In certain embodiments, the methods are used to generate probes for use in immunofluorescence studies.

### BRIEF DESCRIPTIONS OF THE DRAWINGS

FIG. 1. Schematic for generating chemically ligateable probes from reverse transcription. In the Figure, N refers to any DNA nucleotide (A, T, G, or C), and IN refers to RNA nucleobases (A, U, G, or C), functional group modification refers to any functional group which can participate in chemical ligation such as alkyne, azide, amine, etc. Alkaline hydrolysis removes the in vitro transcribed RNA as well as the RNA nucleobases in the primer, leaving single stranded DNA probes.

FIG. 2. A 4% MetaPhor Agarose Gel shows the full-length product of single stranded DNA probes generated through the described method. Primer 1 contains a 3′ modified alkyne functional groups while Primer 2 contains internally modified alkyne functional groups. The DNA ultramers serve as the length control.

FIG. 3. An alkyne-single stranded DNA (ssDNA) probe generated through the described method, yield bright fluorescent FISH dots. “Click” chemistry was successfully performed on the probes to chemically ligate them in situ. Subsequent harsh washes with 70% formamide at 37° C., followed by rehybridization of the readout demonstrates that the probes are indeed chemically ligated around the RNA molecules and would not dissociate from the harsh treatment.

FIG. 4. EDC 1-ethyl-3-(3-dimethylaminopropyl)-ligateable probes can be generated through the described method. Non-EDC treatment results in loss of signals after harsh.

FIG. 5. EDC-ligateable probes allow in situ EDC crosslinking, and the probes survive harsh formamide wash as examined by rehybridization of the same readouts.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

Unless otherwise noted, terms are to be understood according to conventional usage by those of ordinary skill in the relevant art.

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges.

Oligonucleotides can be of various lengths. In particular embodiments, oligonucleotides can range from about 2 to about 200 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to for a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow: blood: blood cells: ascites: tissue or fine needle biopsy samples: cell-containing body fluids: free floating nucleic acids: sputum: saliva: urine: cerebrospinal fluid, peritoneal fluid: pleural fluid: feces: lymph: gynecological fluids: skin swabs: vaginal swabs: oral swabs; nasal swabs: washings or lavages such as a ductal lavages or broncheoalveolar lavages; aspirates: scrapings: bone marrow specimens: tissue biopsy specimens: surgical specimens; feces, other body fluids, secretions, and/or excretions: and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells.

As disclosed herein, the term “barcode” generally refers to a nucleotide sequence of a label produced by methods described herein. The barcode sequence typically is of a sufficient length and uniqueness to identify a single cell that comprises a molecular target.

As disclosed herein, the term “cross-linked” means that a covalent bond is formed between two molecules. In certain embodiments, “cross-link” may be in cis (between the same molecule) or trans (between different molecules).

As disclosed herein, the term “nucleotide” refers to a deoxyribonucleotide, a derivative of a deoxyribonucleotide, a ribonucleotide, or a derivative of a ribonucleotide.

As disclosed herein, the term “350ctdU” refers to co5-Octadiynyl dU, which is a modified base with an eight carbon linker terminating in an alkyne group. A person of skill would recognize that this modified base is one way to insert alkynes at internal positions within a sequence.

As disclosed herein, the term “buffer” refers to refers to a solution containing a buffering agent or a mixture of buffering agents and, optionally, a divalent cation and a monovalent cation.

As disclosed herein, the term “reverse transcription reaction mixture” refers to an aqueous solution comprising the various reagents used to reverse transcribe an RNA template. In certain embodiments, the reverse transcription reaction mixture comprises enzymes, aqueous buffers, salts, oligonucleotide primers, RNA template, and nucleoside triphosphates.

As disclosed herein, the term “molecular targets” refers to DNA, RNA, protein, lipids, glycans, cellular targets, organelles, or any combinations thereof.

### DESCRIPTION

In this disclosure, methods are presented to massively generate chemically ligateable probes with a DNA:RNA hybrid primer containing one or more moieties capable of chemical ligation internally or at the 3′ end of the sequences through reverse transcription.

In some embodiments, a method to generate probes comprises contacting one or more RNA templates with a reverse transcriptase and one or more hybrid-primers under conditions suitable for reverse transcription. In certain embodiments, the hybrid-primer hybridizes to at least one of the one or more RNA templates.

### RNA Templates

In some embodiments, the RNA templates comprise templates isolated from biological samples. In some embodiments, RNA templates comprise a population of heterogeneous RNA molecules in a sample. In some embodiments, the RNA templates comprise a specific RNA molecule. In some embodiments, the RNA templates comprise RNA indicative of a specific disease or infectious agent.

In some embodiments, RNA templates are selected from synthetic RNA, RNA generated from natural or synthetic DNA, transcripts, mRNA, rRNA, tRNA, snRNA, long non-coding RNA (lncRNA), microRNA (miRNA), short interfering RNA (siRNA), piwi-interacting RNA (piRNA), small nucleolar RNA (snoRNA), other short RNAs, and any combinations thereof.

In some embodiments, the RNA templates comprises synthetic RNA. In some embodiments, the RNA template is generated from from natural or synthetic DNA, transcripts. In some embodiments, the RNA templates comprise an mRNA. In some embodiments, RNA templates comprise rRNA. In some embodiments, the RNA templates comprise non-coding RNA.

In some embodiments, the RNA template is transcribed from a DNA template. In certain embodiments, the DNA template comprises oligonucleotides. In certain embodiments, the DNA template comprises a T7 RNA transcriptase promoter. In certain embodiments, the DNA template comprises synthetic DNA complex pools (ssDNA sequences).

In some embodiments, the DNA template is amplified by polymerase chain reaction (PCR) before in vitro transcription to generate the RNA templates.

### Hybrid Primer

In some embodiments, the method comprises using a hybrid primer. In some embodiments, the hybrid-primer comprises one or more deoxyribonucleotides, one or more ribonucleotides, and one or more reactive groups at the 3′ end of the primer.

In some embodiments, the hybrid-primers are at least 10 nucleotides long. In some embodiments, the hybrid-primers are at least 11 nucleotides long. In some embodiments, the hybrid-primers are at least 12 nucleotides long. In some embodiments, the hybrid-primers are at least 13 nucleotides long. In some embodiments, the hybrid-primers are at least 14 nucleotides long. In some embodiments, the hybrid-primers are at least 15 nucleotides long. In some embodiments, the hybrid-primers are at least 16 nucleotides long. In some embodiments, the hybrid-primers are at least 17 nucleotides long. In some embodiments, the hybrid-primers are at least 18 nucleotides long. In some embodiments, the hybrid-primers are at least 19 nucleotides long. In some embodiments, the hybrid-primers are at least 20 nucleotides long. In some embodiments, the hybrid-primers are at least 21 nucleotides long. In some embodiments

In some embodiments, the hybrid-primers comprise at least 1 deoxyribonucleotide. In some embodiments, the hybrid-primers comprise at least 2 deoxyribonucleotides. In some embodiments, the hybrid-primers comprise at least 3 deoxyribonucleotides. In some embodiments, the hybrid-primers comprise at least 4 deoxyribonucleotides. In some embodiments, the hybrid-primers comprise at least 5 deoxyribonucleotides. In some embodiments, the hybrid-primers comprise at least 6 deoxyribonucleotides. In some embodiments, the hybrid-primers comprise at least 7 deoxyribonucleotides. In some embodiments, the hybrid-primers comprise at least 8 deoxyribonucleotides. In some embodiments, the hybrid-primers comprise at least 9 deoxyribonucleotides. In some embodiments, the hybrid-primers comprise at least 10 deoxyribonucleotides. In some embodiments, the hybrid-primers comprise at least 11 deoxyribonucleotides. In some embodiments, the hybrid-primers comprise at least 12 deoxyribonucleotides. In some embodiments, the hybrid-primers comprise at least 13 deoxyribonucleotides. In some embodiments, the hybrid-primers comprise at least 14 deoxyribonucleotides. In some embodiments, the hybrid-primers comprise at least 15 deoxyribonucleotides. In some embodiments, the hybrid-primers comprise at least 16 deoxyribonucleotides. In some embodiments, the hybrid-primers comprise at least 17 deoxyribonucleotides. In some embodiments, the hybrid-primers comprise at least 18 deoxyribonucleotides. In some embodiments, the hybrid-primers comprise at least 19 deoxyribonucleotides. In some embodiments, the hybrid-primers comprise at least 20 deoxyribonucleotides. In some embodiments, the hybrid-primers comprise at least 21 deoxyribonucleotides.

In some embodiments, the hybrid-primers comprise at least 1 ribonucleotide. In some embodiments, the hybrid-primers comprise at least 2 ribonucleotides. In some embodiments, the hybrid-primers comprise at least 3 ribonucleotides. In some embodiments, the hybrid-primers comprise at least 4 ribonucleotides. In some embodiments, the hybrid-primers comprise at least 5 ribonucleotides. In some embodiments, the hybrid-primers comprise at least 6 ribonucleotides. In some embodiments, the hybrid-primers comprise at least 7 ribonucleotides. In some embodiments, the hybrid-primers comprise at least 8 ribonucleotides. In some embodiments, the hybrid-primers comprise at least 9 ribonucleotides. In some embodiments, the hybrid-primers comprise at least 10 ribonucleotides. In some embodiments, the hybrid-primers comprise at least 11 ribonucleotides. In some embodiments, the hybrid-primers comprise at least 12 ribonucleotides. In some embodiments, the hybrid-primers comprise at least 13 ribonucleotides. In some embodiments, the hybrid-primers comprise at least 14 ribonucleotides. In some embodiments, the hybrid-primers comprise at least 15 ribonucleotides. In some embodiments, the hybrid-primers comprise at least 16 ribonucleotides. In some embodiments, the hybrid-primers comprise at least 17 ribonucleotides. In some embodiments, the hybrid-primers comprise at least 18 ribonucleotides. In some embodiments, the hybrid-primers comprise at least 19 ribonucleotides. In some embodiments, the hybrid-primers comprise at least 20 ribonucleotides. In some embodiments, the hybrid-primers comprise at least 21 ribonucleotides.

In some embodiments, the hybrid-primers comprise at least 1 deoxyribonucleotide and at least 2 ribonucleotides. In some embodiments, the hybrid-primers comprise at least 2 deoxyribonucleotides and at least 2 ribonucleotides. In some embodiments, the hybrid-primers comprise at least 3 deoxyribonucleotides and at least 2 ribonucleotides. In some embodiments, the hybrid-primers comprise at least 4 deoxyribonucleotides and at least 2 ribonucleotides. In some embodiments, the hybrid-primers comprise at least 5 deoxyribonucleotides and at least 2 ribonucleotides. In some embodiments, the hybrid-primers comprise at least 6 deoxyribonucleotides and at least 2 ribonucleotides. In some embodiments, the hybrid-primers comprise at least 7 deoxyribonucleotides and at least 2 ribonucleotides. In some embodiments, the hybrid-primers comprise at least 8 deoxyribonucleotides and at least 2 ribonucleotides. In some embodiments, the hybrid-primers comprise at least 9 deoxyribonucleotides and at least 2 ribonucleotides. In some embodiments, the hybrid-primers comprise at least 10 deoxyribonucleotides and at least 2 ribonucleotides.

In some embodiments, the hybrid-primer comprises ribonucleotides spaced among the deoxynucleotides so that after the RNA template is degraded, oligonucleotides are produced. In certain embodiments, the hybrid-primer comprises ribonucleotides spaced in between every other deoxynucleotides.

In some embodiments, the hybrid-primer comprises a sequence complementarity to a region of the RNA template that is at least 60%, 65%, 70%, 75%, 80%, 85%, 90%, 91%, 92%, 93%, 94%, 95%, 96%, 97%, 98%, 99%, or 100%.

### Reactive Groups

In some embodiments, the hybrid primers comprise reactive groups. In some embodiments, the reactive group is selected from alkyne, azide, amide, nitrone, alkene, tetrazine, tetrazole, carboxyl, carbodiimide, amine, phosphoryl, NHS ester, and click chemistry reactive pair members.

In some embodiments, the hybrid-primers comprise at least 1 reactive group. In some embodiments, the hybrid-primers comprise at least 2 reactive groups. In some embodiments, the hybrid-primers comprise at least 3 reactive groups. In some embodiments, the hybrid-primers comprise at least 4 reactive groups. In some embodiments, the hybrid-primers comprise at least 5 reactive groups.

In some embodiments, the hybrid-primer of any of the previous embodiments, has ribonucleotides spaced throughout the primer. In certain embodiments, the hybrid-primer of any of the previous embodiments comprises a ribonucleotide 5′ to a reactive group, the reactive group at the 3′ end. In certain embodiments, the hybrid-primer of any of the previous embodiments, has a ribonucleotide immediately 5′ of the reactive group.

In some embodiments, the probes are cross-linked to molecular targets. In certain embodiments, the molecular targets comprise DNA, RNA, protein, lipids, glycans, cellular targets, organelles, or any combinations thereof. In certain embodiments, the reactive groups on the probes crosslink with amines on the molecular targets. In certain embodiments, the probes are cis-cross-linked 5′ to 3′ with click chemistry.

### Reverse Transcription

In some embodiments, the method comprises reverse transcribing the RNA template.

In some embodiments, the RNA template is purified before reverse transcription. In certain embodiments, RNA template is purified by ion exchange chromatography, magnetic beads that selectively bind the RNA template, phenol-chloroform extraction, ethanol precipitation, or any combination thereof.

In some embodiments, the method comprises a reverse transcription reaction mixture. In certain embodiments, the reverse transcription reaction mixture comprises an aqueous solution comprising the various reagents used to reverse transcribe an RNA template. In certain embodiments, the reverse transcription reaction mixture comprises enzymes, aqueous buffers, salts, oligonucleotide primers, RNA template, and deoxyribonucleotide triphosphate (dNTPs). In certain embodiments, the conditions suitable for reverse transcription further comprise a pool of nucleotides for the reverse transcriptase.

In some embodiments, the conditions suitable for reverse transcription further comprise buffer conditions for the reverse transcriptase to function.

In some embodiments, the reverse transcriptase is Maxima H minus reverse transcriptase, Superscript IV reverse transcriptase, M-MuLV reverse transcriptase, or AMV Reverse Transcriptase. In certain embodiments, the reverse transcriptase is Maxima H minus reverse transcriptase.

In some embodiments, the conditions comprise 10-100 mM Tris-HCL pH 7.5-8.5 at 25° C., 1-10 mM MgCl₂, 1-50 mM DTT, 25-100 mM KCL, 0.1-1 mM deoxyribonucleotide triphosphate (dNTP), and 0.1-5.0 units of enzyme. In certain embodiments, the conditions comprise heating the reverse transcriptase reaction at 37° C. In certain embodiments, the conditions comprise 50 mM Tris-HCl (pH 8.3 at 25° C.), 75 mM KCl, 3 mM MgCl₂, 10 mM DTT).

In some embodiments, the conditions suitable for reverse transcription further comprises a temperature for the reverse transcriptase to transcribe. In some embodiments, the temperature comprises 25, 30, 35, 37, 45, 50, 55, 60, 65, or 70° C.

### Degrading the RNA Template

In some embodiments, the method comprises degrading the RNA template.

In some embodiments, the RNA template is degraded by alkaline hydrolysis. In certain embodiments, the RNA template is degraded by adding 0.1-4M KOH to the template. In certain embodiments, the RNA template is degraded by heating the template with KOH at a temperature comprising 25, 30, 35, 37, 45, 50, 55, 60, 65, or 70° C. In certain embodiments, the RNA template is degraded by heating the template for at least 0.1, 0.2, 0.3, 0.4, 0.5, 1, 2, 3, 4, 5, 6, 7, 8, 9, 10, 11, 12, 13, 14, 15, 16, 17, 18, 19, or 20 hours. In certain embodiments, the method comprises degrading the RNA template by treating the RNA template with 0.25 M NaOH at 65° C. for at least 0.3 hours. In certain embodiments, the method comprises degrading the RNA template by treating the RNA template with 0.25 M NaOH at 90° C. for at least 0.16 hours.

In some embodiments, the RNA template is degraded by enzymatic degradation. In certain embodiments, the enzymatic degradation is by an RNase. In certain embodiments, the RNase is selected from RNase A, RNAse H, or any combination thereof.

### Generating the Probes

In some embodiments, the method comprises generating single stranded DNA probes by RNA degradation. In certain embodiments, the probes produced after RNA degradation comprise at least one nucleotide in length. In certain embodiments, the probes produced after RNA degradation comprise at least two nucleotides in length. In certain embodiments, the probes produced by RNA degradation comprise at least three nucleotides in length. In certain embodiments, the probes produced after RNA degradation comprise at least four nucleotides in length. In certain embodiments, the probes produced by RNA degradation comprise at least five nucleotides in length. In certain embodiments, the probes produced after RNA degradation comprise at least six nucleotides in length. In certain embodiments, the probes produced after RNA degradation comprise at least seven nucleotides in length. In certain embodiments, the probes produced after RNA degradation comprise at least eight nucleotides in length. In certain embodiments, the probes produced after RNA degradation comprise at least nine nucleotides in length. In certain embodiments, the probes produced after RNA degradation comprise at least 10 nucleotides in length. In certain embodiments, the probes produced after RNA degradation comprise at least 15 nucleotides in length. In certain embodiments, the probes produced after RNA degradation comprise at least 20 nucleotides in length. In certain embodiments, the probes produced after RNA degradation comprise at least 25 nucleotides in length. In certain embodiments, the probes produced after RNA degradation comprise at least 30 nucleotides in length. In certain embodiments, the probes produced after RNA degradation comprise at least 35 nucleotides in length. In certain embodiments, the probes produced after RNA degradation comprise at least 40 nucleotides in length. In certain embodiments, the probes produced after RNA degradation comprise at least 45 nucleotides in length. In certain embodiments, the probes produced after RNA degradation comprise at least 50 nucleotides in length. In certain embodiments, the probes produced after RNA degradation comprise at least 100 nucleotides in length. In certain embodiments, the probes produced after RNA degradation comprise at least 200 nucleotides in length. In certain embodiments, the probes produced after RNA degradation comprise at least 500 nucleotides in length. In certain embodiments, the probes produced after RNA degradation comprise at least 1000 nucleotides in length.

In some embodiments, the probes of any of the preceding embodiments comprises oligonucleotides that are less than 30, 50, 100, 200, 250, 500, 750, or 1000 nucleotides in length.

### Washing the Probes

In some embodiments, the method comprises washing the probes after each step. In some embodiments, the method comprises washing the probes with a buffer that removes non-specific interactions. In some embodiments, the method comprises washing the probes with a buffer that removes non-specific hybridization reactions. In some embodiments, the buffer is stringent. In certain embodiments, the wash buffer comprises 10% formamide, 2×SSC, and 0.1% triton X-100s. In certain embodiments, the wash buffer comprises 20% formamide, 2×SSC, and 0.1% triton X-100s. In certain embodiments, the wash buffer comprises 30% formamide, 2×SSC, and 0.1% triton X-100s. In certain embodiments, the wash buffer comprises 40% formamide, 2×SSC, and 0.1% triton X-100s. In certain embodiments, the wash buffer comprises 50% formamide, 2×SSC, and 0.1% triton X-100s.

### Isolating the Probes

In some embodiments, the method comprises isolating the probe. In some embodiments, the method comprises isolating the one or more probes by column chromatography, gel filtration, precipitation techniques, or any combination thereof. In some embodiments, the method comprises isolating the probes by Polyacrylamide gel electrophoresis (PAGE). In some embodiments, the method comprises Reverse Phase High Pressure Liquid Chromatography (RP HPLC). In some embodiments, the method comprises Anion Exchange High Pressure Liquid Chromatography.

In some embodiments, the method comprises isolating one or more single stranded DNA probes with at least of the one or more reactive groups at its 5′ end.

In some embodiments, the method comprises isolating one or more single stranded DNA probes of any of the previous embodiments, with at least one or more reactive groups at its 3′ end.

### Using the Probes

In some embodiments, the probes are used to label one or more molecular targets. In some embodiments, the probes are cross-linked to one or more molecular targets. In some embodiments, the probes are used in methods to determine the interaction of molecular targets with each other. In some embodiments, the probes are used for cell labeling experiments. In some embodiments, the probes are used to determine the biological mechanisms of the molecular targets. In some embodiments, the probes are used for cell tracking experiments. In certain embodiments, the cell tracking experiments distinguish transplanted cells from host cells, monitor distribution, migration after transplantation, evaluate functional effects of the transplanted cells, or any combination thereof.

In some embodiments, the probes are used in a method to barcode one or more molecular targets. See, for example, International PCT Patent Application Publication No. WO2014182528A2, filed Apr. 30, 2014 as International Patent Application No. PCT/US2014/036258 titled MULTIPLEX LABELING OF MOLECULES BY SEQUENTIAL HYBRIDIZATION BARCODING IDENTIFICATION, the entire contents of which are herein incorporated by reference in its entirety for all purposes.

In some embodiments, the probes are used in a method for linked amplification tethered with exponential radiance (LANTERN). See, for example, International Patent Application No. PCT/US2022/021826, FILED Mar. 24, 2022, and titled LINKED AMPLIFICATION TETHERED WITH EXPONENTIAL RADIANCE, the entire contents of which are herein incorporated by reference in its entirety for all purposes.

In some embodiments, the probes are cross-linked 5′ to 3′ for use in LANTERN experiments. In some embodiments, the probes are cis cross-linked 5′ to 3′ for use in LANTERN experiments. In some embodiments, the probes are cis cross-linked 5′ to 3′ with click chemistry for use in LANTERN experiments.

In some embodiments, the probes are used in ClampFISH experiments. See, for example, ClampFISH detects individual nucleic acid molecules using click chemistry-based amplification, Rouhanifard S. H. et al., Nature Biotechnology 37: 84-89 (2019), the entire contents of which are herein incorporated by reference in its entirety for all purposes.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### EXAMPLES

### Example 1

An exemplary Example of primer sequences includes:

(1)
GCCCCrATCArUGTGrCCTTTCrC /350ctdU/
and
(2)
GCCCCrATCArUGTGCCTrU/i5OctdU/CCT.

In some embodiments, a primer sequence is represented by a formula that includes basepairs, wherein N represents a DNA nucleobase, IN represents RNA nucleobase, and containing moiety is an alkyne functional group placed at the 3′ end or internally.

### Example 2

To test whether a reverse transcriptase can utilize the hybrid primer from Example 1, reverse transcribing the RNA into single stranded DNA sequences was tested.

PCR was performed on the DNA templates from oligo complex pool to generate RNA through in vitro transcription. Alternatively, direct sequences such as those synthesized from IDT oPools containing T7 sequences for in vitro transcription were used.

RNA templates were then reverse transcribed with reverse transcriptase, together with the DNA:RNA hybrid primer containing a moiety capable of chemical ligation. In a typical reaction, 5 nmoles of RNA template was mixed with 1.5× more of the hybrid primer and 5 μL of reverse transcriptase (1000 unit), 3 uM of each dNTP, and 1:100 of an RNase inhibitor.

The RNA as well as the RNA nucleobases in the final single stranded DNA (ssDNA) was degraded by alkaline hydrolysis using sodium hydroxide.

If necessary, the 3′ end of the primary probes were incorporated by terminal transferase with nucleotides containing a moiety capable of chemical ligation with the 5′ functional group. For example, TdT was incorporated N6-(6-Azido)hexyl-dATP so that the probes were “click” ligated with the 5′ alkyne when hybridized as padlock probes.

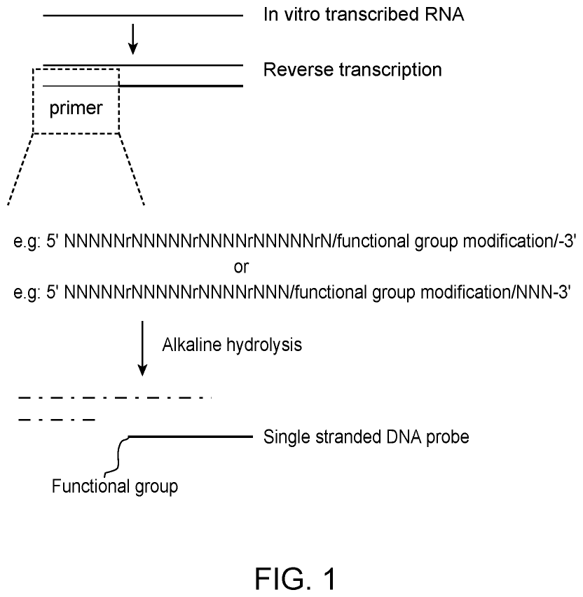

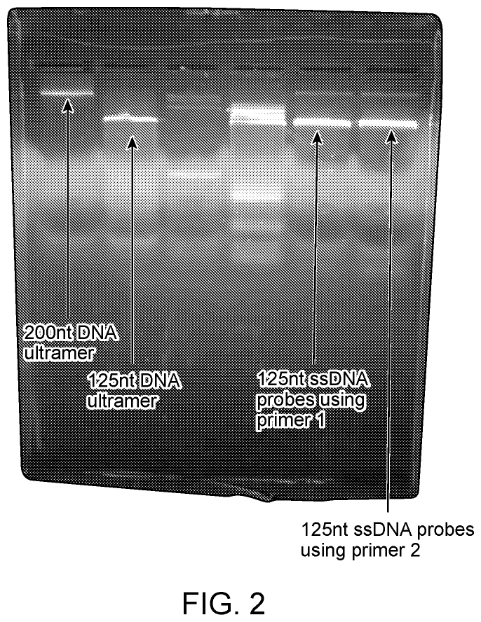

Current chemically ligateable probes are synthesized by ligation which limits the throughput. Here, we generated thousands of chemically ligateable probes by reverse transcribing those RNA sequences into single stranded DNA probes by using a DNA: RNA hybrid primer containing the moiety for chemical ligation (FIG. 1, FIG. 2).

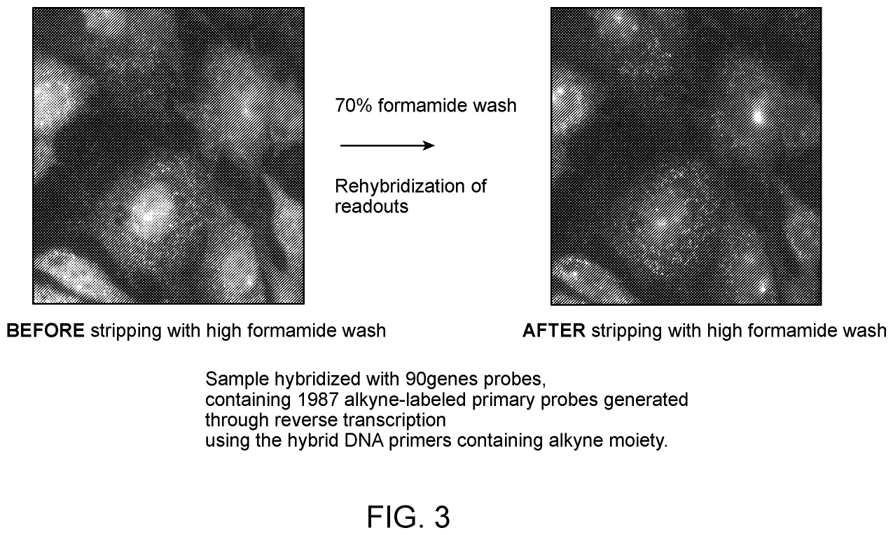

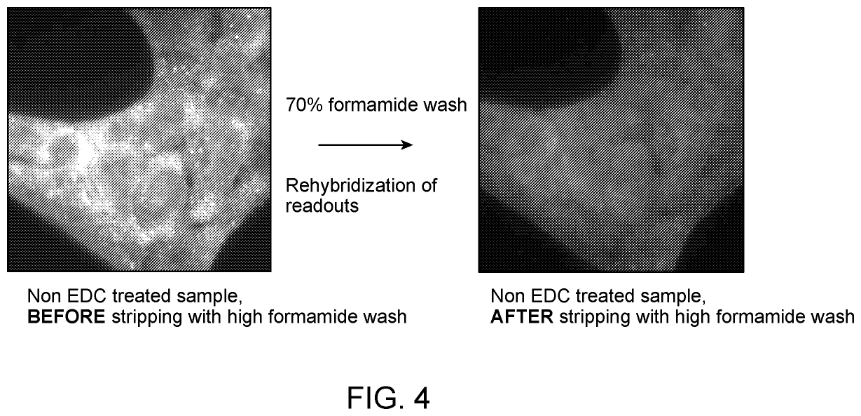

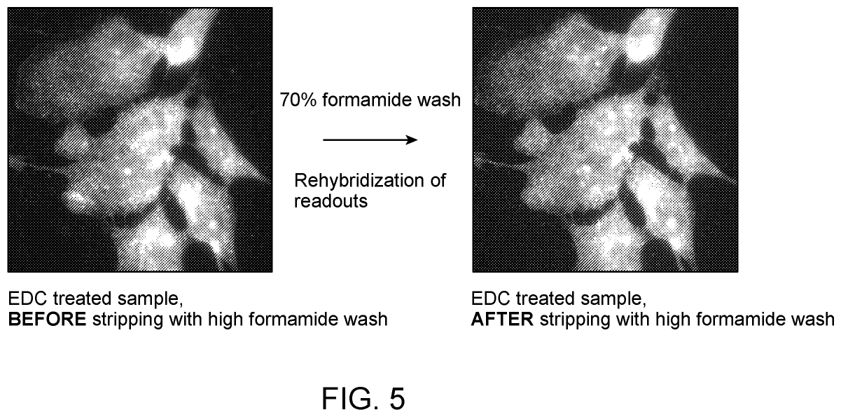

The probes were tested by hybridizing to RNA in fixed samples and showed that they produced bright fluorescent dots. Thousands of chemically ligateable probes were generated through this method. Further, the probes were functional in generating bright fluorescent dots in highly multiplexed FISH experiments. Harsh stripping of the probes and rehybridization showed that the probes are functional in chemical ligation, as signals are retained (FIG. 3). The same strategy of probe generation was used to generate probes containing 5′ phosphate and 3′ amine and could be used to the probes in situ through EDC (1-ethyl-3-(3-dimethylaminopropyl) carbodiimide hydrochloride) crosslinking chemistry (FIG. 4 and FIG. 5).

### REFERENCES

The following references are incorporated by their entirety.
Murgha, Y., Beliveau, B., Semrau, K., Schwartz, D., Wu, C.- T., Gulari, E., & Rouillard, J.- M. (2015). Combined in vitro transcription and reverse transcription to amplify and label complex synthetic oligonucleotide probe libraries. BioTechniques, 58(6), 301-307.
Murgha, Y. E., Rouillard, J.- M., & Gulari, E. (2014). Methods for the preparation of large quantities of complex single-stranded oligonucleotide libraries. PloS One, 9(4), e94752.
Randolph, J. B., and Waggoner, A. S. Stability, specificity and fluorescence brightness of multiply-labeled fluorescent DNA probes. (1997) Nucleic Acids Res 25(14), 2923-9.
Brumbaugh, J. A., Middendorf, L. R., Grone, D. L., and Ruth, J. L. Continuous, on-line DNA sequencing using oligodeoxynucleotide primers with multiple fluorophores. (1988) Proc Natl Acad Sci USA 85(15), 5610-4.
Hughes, T. R. et al. Expression profiling using microarrays fabricated by an ink-jet oligonucleotide synthesizer. (2001) Nat Biotechnol 19(4), 342-7.
U.S. Ser. No. 10/457,980B2. Multiplex labeling of molecules by sequential hybridization barcoding.

1. A method to generate probes, the method comprising:
(i) contacting one or more RNA templates with a reverse transcriptase and one or more hybrid-primers under conditions suitable for reverse transcription, wherein each hybrid-primer hybridizes to at least one of the one or more RNA templates, and wherein each hybrid-primer comprises one or more of each of:
(a) one or more deoxyribonucleotides;
(b) one or more ribonucleotides; and
(c) one or more reactive groups, at the 3′ end of the primer;
(ii) degrading the RNA template and the hybrid-primer; and
(iii) isolating one or more single stranded DNA probes comprising at least one or more reactive groups at its 5′ end.

2. The method of claim 1, wherein the one or more RNA templates are selected from synthetic RNA, RNA generated from natural or synthetic DNA, transcripts, mRNA, rRNA, tRNA, snRNA, long non-coding RNA (lncRNA), microRNA (miRNA), short interfering RNA (siRNA), piwi-interacting RNA (piRNA), small nucleolar RNA (snoRNA), other short RNAs, and any combinations thereof.

3. The method of claim 1, wherein the reactive group is selected from alkyne, azide, amide, nitrone, alkene, tetrazine, tetrazole, carboxyl, carbodiimide, amine, phosphoryl, NHS ester, and click chemistry reactive pair members.

4. The method of claim 1, wherein the degrading is by alkaline hydrolysis.

5. The method of claim 1, wherein the degrading is by enzymatic degradation.

6. The method of claim 5, wherein the enzymatic degradation is by an RNase.

7. The method of claim 6, wherein the RNase is selected from RNase A, RNAse H, or any combination thereof.

8. The method of claim 1, wherein the conditions suitable for reverse transcription further comprise deoxyribonucleotide triphosphates (dNTPs), buffer conditions, and a suitable temperature for the reverse transcriptase to function.

9. The method of claim 1, wherein each hybrid-primer is at least 17 nucleotides in length.

10. The method of claim 1, wherein the hybrid-primer comprises a sequence complementarity to a region of the RNA template that is at least 60%, 65%, 70%, 75%, 80%, 85%, 90%, 91%, 92%, 93%, 94%, 95%, 96%, 97%, 98%, 99%, or 100%.

11. The method of claim 1, wherein the probes are washed after each step.

12. The method of claim 11, wherein the probes are washed with a buffer that removes non-specific interactions.

13. The method of claim 12, wherein the buffer is stringent.
